(19) United States
(12) Patent Application Publication
(10) Pub. No.: US 2004/0087513 A1
(43) Pub. Date: May 6, 2004

(54) New drug combinations based on sodium channel blockers and magnesium salts

(75) Inventors: Judith Duettmann, Gau-Algesheim (DE); Thomas Weiser, Wiesbaden (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma GmbH & Co. KG, Ingelheim (DE)

(21) Appl. No.: 10/612,107

(22) Filed: Jul. 2, 2003

Related U.S. Application Data
(60) Provisional application No. 60/408,213, filed on Sep. 4, 2002.

(30) Foreign Application Priority Data
Jul. 4, 2002 (DE) 10230027

(51) Int. Cl. A61K 31/7024; A61K 31/4745; A61K 31/55

(52) U.S. Cl. 514/23; 514/295; 514/217; 514/464; 514/459

(57) ABSTRACT

The invention relates to new drug combinations based on sodium channel blockers 1 and magnesium salts 2, processes for the preparation thereof as well as the use thereof for preparing pharmaceutical compositions for the treatment of ischaemic conditions.

### RELATED APPLICATIONS

Benefit of U.S. Provisional Application Serial No. 60/408,213, filed on Sep. 4, 2002 is hereby claimed, and said Application is herein incorporated by reference.

The invention relates to new drug combinations based on sodium channel blockers 1 and magnesium salts 2, processes for the preparation thereof as well as the use thereof for preparing pharmaceutical compositions for the treatment of ischaemic conditions.

### DESCRIPTION OF THE INVENTION

The invention relates to drug combinations containing one or more, preferably one sodium channel blocker 1 and one or more, preferably one magnesium salt 2, optionally in the presence of conventional excipients or carriers.

A) Sodium Channel Blockers 1 Which May be Used According to the Invention:

Within the scope of the present invention preferred sodium channel blockers 1 are those selected from the group consisting of pirmencol, sipatrigine, irampanel, pilsicainide, oxcarbazepine, topiramate, fosphenytoin, flunarizin, ropivacaine, levobupivacaine, zonisamide, mexiletine, bipridil, bisaramil, milacainide, safinamide, bupivacaine, tetrodotoxin, NS 7, the compounds of general formula 1a

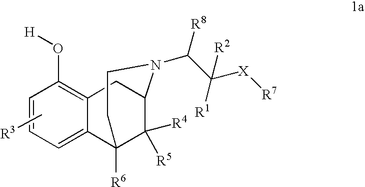

wherein

X denotes a single bond, —O, C₁-C₄-alkylene, an alkylene bridge with 1 to 8 carbon atoms which may be branched or unbranched and may have at any point in the bridge one or two oxygen atom(s) or a nitrogen atom, preferably O—C₁-C₃-alkylene or —O—CH₂—CH₂—O, —O—CH₂—CH₂—NH—;

R¹ denotes hydrogen, methyl, ethyl, phenyl;

R² denotes hydrogen, methyl;

R³ denotes hydrogen, fluorine, chlorine, bromine, hydroxy, methyl, methoxy;

R⁴ denotes hydrogen, methyl, ethyl;

R⁵ denotes hydrogen, methyl, ethyl;

R⁶ denotes hydrogen, methyl, ethyl;

R⁷ denotes tert.-butyl, cyclohexyl or phenyl, while phenyl may optionally be substituted by R⁹ and R¹⁰, which may be identical or different;

R⁸ denotes hydrogen, C₁-C₄-alkyl;

R⁹ denotes hydrogen, methyl, fluorine, chlorine, bromine, methoxy;

R¹⁰ denotes hydrogen, methyl, fluorine, chlorine, bromine, methoxy;

optionally in the form of the individual optical isomers, mixtures of the individual enantiomers or racemates as well as in the form of the free bases or the corresponding acid addition salts with pharmacologically acceptable acids;

and the compounds of general formula 1b.

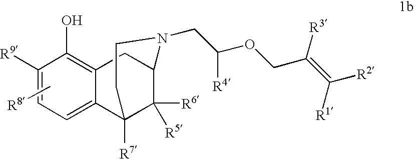

wherein

R^{1′}, R^{2′} and R^{3′} which may be identical or different, denote hydrogen, methyl or ethyl;

R^{4′} denotes hydrogen, methyl or ethyl;

R^{5′}, R^{6′} and R^{7′} which may be identical or different, denote hydrogen, methyl or ethyl;

R^{8′} and R^{9′} which may be identical or different, denote hydrogen, fluorine, chlorine, bromine, methyl, ethyl, hydroxy or methoxy,

optionally in the form of the racemates, the enantiomers, the diastereomers and the mixtures thereof, and optionally the pharmacologically acceptable acid addition salts thereof.

Particularly preferred within the scope of the present invention is or are the sodium channel blocker(s) 1 selected from the group consisting of pirmencol, pilsicainide, sipatrigine, irampanel, fosphenytoin, zonisamide, mexiletine, bipridil, bisaramil, milacainide, NS 7, the compounds of general formula 1a wherein

X denotes C₁-C₃-alkylene, —O—CH₂—CH₂—O— or —O—CH₂—CH₂—NH—;

R¹ denotes hydrogen or methyl;

R² denotes hydrogen or methyl;

R³ denotes hydrogen or chlorine;

R⁴ denotes hydrogen or methyl;

R⁵ denotes hydrogen or methyl;

R⁶ denotes methyl or ethyl;

R⁷ denotes tert.-butyl, cyclohexyl or phenyl, while phenyl may optionally be substituted by R⁹ and R¹⁰, which may be identical or different;

R⁸ denotes hydrogen;

R⁹ denotes hydrogen, methyl, fluorine or chlorine;

R¹⁰ denotes hydrogen, methyl, fluorine or chlorine;

optionally in the form of the individual optical isomers, mixtures of the individual enantiomers or racemates as well as in the form of the free bases or the corresponding acid addition salts with pharmacologically acceptable acids;

and the compounds of general formula 1b, wherein

R^{1′}, R^{2′} and R^{3′} which may be identical or different, denote hydrogen or methyl;

R^{4′} denotes hydrogen or methyl;

R^{5′}, R^{6′} and R^{7′} which may be identical or different, denote hydrogen or methyl, preferably methyl;

R^{8′} denotes hydrogen, methyl, hydroxy or methoxy, preferably hydrogen or methyl,

R^{9′} denotes hydrogen or methyl,

optionally in the form of the racemates, the enantiomers, the diastereomers and the mixtures thereof, and optionally the pharmacologically acceptable acid addition salts thereof.

Particularly preferred within the scope of the present invention is or are the sodium channel blocker(s) 1 selected from the group consisting of fosphenytoin, zonisamide, sipatrigine, irampanel, mexiletine, NS 7, the compounds of general formula 1a wherein

X denotes C₁-C₃-alkylene or —O—CH₂—CH₂—O—;

R¹ denotes hydrogen or methyl;

R² denotes hydrogen or methyl;

R³ denotes hydrogen;

R⁴ denotes hydrogen or methyl;

R⁵ denotes hydrogen or methyl;

R⁶ denotes methyl;

R⁷ denotes phenyl, the phenyl may optionally be substituted by R⁹ and R¹⁰, which may be identical or different;

R⁸ denotes hydrogen;

R⁹ denotes hydrogen, methyl, fluorine or chlorine;

R¹⁰ denotes hydrogen, methyl, fluorine or chlorine;

optionally in the form of the individual optical isomers, mixtures of the individual enantiomers or racemates as well as in the form of the free bases or the corresponding acid addition salts with pharmacologically acceptable acids;

and the compounds of general formula 1b, wherein

R^{1′}, R^{2′} and R^{3′} which may be identical or different, denote hydrogen or methyl;

R^{4′} denotes hydrogen or methyl;

R^{5′}, R^{6′} and R^{7′} denote methyl;

R^{8′} denotes hydrogen or methyl, preferably hydrogen;

R^{9′} denotes hydrogen or methyl,

optionally in the form of the racemates, the enantiomers, the diastereomers and the mixtures thereof, and optionally the pharmacologically acceptable acid addition salts thereof.

C₁-C₄-alkyl or C₁-C₈-alkyl generally denotes a branched or unbranched hydrocarbon group with 1 to 4 or 1 to 8 carbon atom(s), which may optionally be substituted by one or more halogen atoms—preferably fluorine—which may be identical to or different from one another. The following hydrocarbon groups are mentioned by way of example: methyl, ethyl, propyl, 1-methylethyl (isopropyl), n-butyl, 1-methylpropyl, 2-methylpropyl, 1,1-dimethylethyl, pentyl, 1-methylbutyl, 2-methylbutyl, 3-methylbutyl, 1,1-dimethylpropyl, 1,2-dimethylpropyl, 2,2-dimethylpropyl, 1-ethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2,-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl. Unless otherwise stated, lower alkyl groups with 1 to 4 carbon atoms, such as methyl, ethyl, propyl, iso-propyl, n-butyl, 1-methylpropyl, 2-methylpropyl or 1,1-dimethylethyl, are preferred.

Accordingly, alkylene denotes a branched or unbranched double-bonded hydrocarbon bridge with 1 to 8 carbon atoms, which may optionally be substituted by one or more halogen atoms—preferably fluorine—which may be identical to or different from one another.

Alkoxy generally denotes a straight-chain or branched hydrocarbon group bonded via an oxygen atom—a lower alkoxy group with 1 to 4 carbon atom(s) is preferred. The methoxy group is particularly preferred.

A preferred compound of formula 1a is (-)-(1R,2″S)-2-(2″-benzyloxy)propyl-4′-hydroxy-5,9,9-trimethyl-6,7-benzomorphan in the form of the free bases or the corresponding acid addition salts with pharmacologically acceptable acids. This compound is also known by the name crobenetine.

Of particular interest are the following compounds of general formula 1b: (2R)-N-allyloxyethyl-1,2,3,4,5,6-hexahydro-6,11,11-trimethyl-2,6-methano-3-benzazocin-10-ol-hydrochloride and (2R,2″S)-N-(2-allyloxy-propyl )-1,2,3,4,5,6-hexahydro-6,11,11-trimethyl-2,6-methano-3-benzazocin-10-ol-hydrochloride.

Accordingly, within the scope of the present invention, the component 1 is most preferably selected from the group consisting of fosphenytoin, zonisamide, sipatrigine, irampanel, mexiletine, NS 7, crobenetine, (2R)-N-allyloxyethyl-1,2,3,4,5,6-hexahydro-6,11,11-trimethyl-2,6-methano-3-benzazocin-10-ol-hydrochloride and (2R,2″S)-N-(2-allyloxy-propyl)-1,2,3,4,5,6-hexahydro-6,11,11-trimethyl-2,6-methano-3-benzazocin-10-ol-hydrochloride, most preferably crobenetine, (2R)-N-allyloxyethyl-1,2,3,4,5,6-hexahydro-6,11,11-trimethyl-2,6-methano-3-benzazocin-10-ol-hydrochloride and (2R,2″S)-N-(2-allyloxy-propyl )-1,2,3,4,5,6-hexahydro-6,11,11-trimethyl-2,6-methano-3-benzazocin-10-ol-hydrochloride.

The compounds 1 may optionally be used in the form of their salts, and, particularly for pharmaceutical use, in the form of the pharmacologically acceptable acid addition salts with an inorganic or organic acid. Suitable acids for this include for example succinic acid, hydrobromic acid, acetic acid, fumaric acid, maleic acid, methanesulphonic acid, lactic acid, phosphoric acid, hydrochloric acid, sulphuric acid, tartaric acid or citric acid. It is also possible to use mixtures of the above acids.

The compounds of formula 1a are known from WO 99/14199. The compounds of formula 1b are not yet known in the prior art.

The compounds of formula 1b which may be used according to the invention and are not yet known in the prior art may be prepared analogously to methods of synthesis known per se. Possible methods of synthesising the compounds of formula 1b are described by way of example hereinafter.

### Synthesis Example 1

### (2R)-N-allyloxyethyl-1,2,3,4,5,6-hexahydro-6,11,11-trimethyl-2,6-methano-3-benzazocin-10-ol-hydrochloride

1.8 g of allyloxyacetic acid are placed in 15 ml dichloromethane, combined with 4.8 g of TBTU and 7.5 ml of ethyldiisopropylamine and stirred at RT for 15 min. Then the mixture is cooled to −5° C. and 3.1 g of 1,2,3,4,5,6-hexahydro-6,11,11-trimethyl-2,6-methano-3-benzazocin-10-ol are added. The mixture is stirred for 30 min at 0° C., and 1 h at RT. Then it is washed once with 100 ml of 2N HCL and once with 100 ml 10% potassium carbonate solution, dried and evaporated down in vacuo. The residue is taken up in 50 ml of THF and added dropwise under nitrogen to a suspension of 1.0 g of lithium aluminium hydride in 50 ml THF. (Temp. rises to 35° C.). It is then heated to 50° C., stirred for 1 h, cooled and at 0-10° C. 1 ml of water is added dropwise, the mixture is stirred for 30 min, 3 ml of NaOH are added and the mixture is stirred for 30 min. The precipitate is suction filtered, the mother liquor evaporated down in vacuo and the residue is filtered through a short column ( approx. 75 ml silica gel; dichloromethane 70, ethyl acetate 20, methanol 10). The appropriate fractions are evaporated down in vacuo and crystallised from acetone+eth.HC1.

Yield 2.8 g of (77%), melting point: 236° C.; [α]_{D}²⁰=−78.3° (c=1; methanol).

### Synthesis Example 2

### (2R,2″S)-N-(2-allyloxy-propyl)-1,2,3,4,5,6-hexahydro-6,11,11-trimethyl-2,6-methano-3-benzazocin-10-ol-hydrochloride

This is prepared analogously to the method according to Example 1. Yield 56%, melting point: 239° C.; [α]_{D}²⁰=−33.9° (c=1; methanol).

### Synthesis Example 3

### (2R,2″S)-N-(2-but-2-enoxy-propyl)-1,2,3,4,5,6-hexahydro-6,11,11-trimethyl-2,6-methano-3-benzazocin-10-ol-hydrochloride

This is prepared analogously to the method according to Example 1.

Yield 47%, melting point: 205° C.

### Synthesis Example 4

### (2R,2″S)-N-[2-(2-methyl-propenoxy)-propyl]-1,2,3,4,5,6-hexahydro-6,11,11-trimethyl-2,6-methano-3-benzazocin-10-ol-hydrochloride

This is prepared analogously to the method according to Example 1.

Yield 12%, melting point: 240° C.; [α]_{D}²⁰=−29.6° (c=1; methanol).

### Synthesis Example 5

### (2R)-N-[2-allyloxy-propyl]-1,2,3,4,5,6-hexahydro-6,11,11-trimethyl-2,6-methano-3-benzazocin-10-ol-hydrochloride

1.9 g of (2R)-N-allyloxyethyl-1,2,3,4,5,6-hexahydro-6,11,11-trimethyl-2,6-methano-3-benzazocin-10-ol-hydrochloride (Example 1) are dissolved in 40 ml of methanol and combined with 3 g of 30% formalin solution and 3 ml of 4 N NaOH. The mixture is heated to 50° C. for 12 hours, the solvent is removed in vacuo, 100 ml of water are added to the residue and it is extracted twice with 200 ml of ether. The organic phase is washed with water, dried and evaporated down in vacuo. The residue is dissolved in 20 ml of dichloromethane and at RT 1.5 g of SOCl₂ added dropwise. After 30 min. it is evaporated down in vacuo, the residue is taken up in 20 ml THF and under nitrogen added dropwise to a suspension of 0.5 g of lithium aluminium hydride in 20 ml of tetrahydrofuran. Then it is heated to 50° C. for 2 h, cooled, 1.5 ml of 4N NaOH are added dropwise and the mixture is stirred for 30 min. The precipitate is suction filtered and the mother liquor is evaporated down in vacuo. The residue is filtered through a short silica gel column (approx 30 ml of silica gel, approx 250 ml of ethyl acetate). ). The appropriate fractions are evaporated down in vacuo and crystallised from acetone+eth.HCl.

Yield 1.1 g of (56%), melting point: 212° C., [α]_{D}²⁰=−71.6° (c=1; methanol).

### Synthesis Example 6

### (2R,2″S)-N-[2-allyloxy-propyl]-1,2,3,4,5,6-hexahydro-6,9,11,11-tetramethyl-2,6-methano-3-benzazocin-10-ol-hydrochloride

This is prepared analogously to the method according to Example 5 starting from Example 2.

Yield 60%, melting point: 215° C.; [α]_{D}²⁰=−29.3° (c=1; methanol).

B) Magnesium Salts 2 Which May be Used According to the Invention:

Within the scope of the present invention the magnesium salts 2 selected from the group consisting of magnesium adipate, magnesium-L-aspartate, magnesium carbonate, magnesium-L-hydrogen aspartate, magnesium hydrogen citrate, magnesium hydrogen glutamate, magnesium sulphate, magnesium chloride, trimagnesium dicitrate and magnesium acetate are preferred.

Particularly preferred within the scope of the present invention are the magnesium salts 2 selected from the group consisting of magnesium sulphate, magnesium chloride and magnesium acetate, while magnesium sulphate is of particular importance according to the present invention.

C) Use of the Drug Combinations of 1 and 2 According to the Invention:

The present invention further relates to the use of the combinations according to the invention of one or more, preferably one sodium channel blocker 1 and one or more, preferably one magnesium salt 2 for preparing a pharmaceutical composition for the treatment of ischaemic conditions of various origins. Preferably, the present invention relates to the use of the combinations according to the invention of one or more, preferably one sodium channel blocker 1 and one or more, preferably one magnesium salt 2 for preparing a pharmaceutical composition for the treatment of cardiac or cerebral ischaemias, most preferably for the treatment of stroke and traumatic brain damage. Of particular importance within the scope of the present invention is the use of the combinations according to the invention of one or more, preferably one sodium channel blocker 1 and one or more, preferably one magnesium salt 2 for the treatment of ischaemic stroke, most preferably acute ischaemic stroke.

The present invention further relates to a process for treating ischaemic conditions of various origins which is characterised in that a combination according to the invention of one or more, preferably one sodium channel blocker 1 and one or more, preferably one magnesium salt 2 is administered. The present invention preferably relates to a method of treating cardiac or cerebral ischaemias, most preferably stroke, and more preferably according to the invention ischaemic stroke, most preferably acute ischaemic stroke, which is characterised in that a combination according to the invention of one or more, preferably one sodium channel blocker 1 and one or more, preferably one magnesium salt 2 is administered.

The present invention further relates to the use of one or more, preferably one sodium channel blocker 1 for preparing a pharmaceutical composition for the combined treatment of ischaemic conditions of various origins with one or more, preferably one magnesium salt 2. The present invention preferably relates to the abovementioned use for preparing a pharmaceutical composition for the combined treatment of cardiac or cerebral ischaemias, most preferably for the treatment of stroke and traumatic brain damage with one or more, preferably one magnesium salt 2. Of particular importance within the scope of the present invention is the present use for the combined treatment of ischaemic stroke, most preferably acute ischaemic stroke with one or more, preferably one magnesium salt 2.

The present invention further relates to a method of treating ischaemic conditions of various origins which is characterised in that one or more, preferably one sodium channel blocker 1 and one or more, preferably one magnesium salt 2 are administered simultaneously or sequentially in one single or two separate, preferably in two separate preparations. The present invention preferably relates to a method of treating cardiac or cerebral ischaemias, most preferably stroke and traumatic brain damage, and more preferably according to the invention ischaemic stroke, most preferably acute ischaemic stroke, which is characterised in that one or more, preferably one sodium channel blocker 1 and one or more, preferably one magnesium salt 2 are administered simultaneously or sequentially in one single or two separate preparations.

D.1) Administration of the Drug Combinations of 1 and 2 According to the Invention:

The drug combinations according to the invention may contain the active ingredients 1 and 2 in one single or two separate preparations. In the combinations of zonisamide, mexiletine, NS 7, crobenetine, (2R)-N-allyloxyethyl-1,2,3,4,5,6-hexahydro-6,11,11-trimethyl-2,6-methano-3-benzazocin-10-ol-hydrochloride or (2R,2″S)-N-(2-allyloxy-propyl)-1,2,3,4,5,6-hexahydro-6,11,11-trimethyl-2,6-methano-3-benzazocin-10-ol-hydrochloride as component 1 with magnesium sulphate, magnesium chloride or magnesium acetate as component 2 which are of particular importance according to the invention, the two components are preferably contained in one or two separation preparations. Separate preparations may also be referred to as so-called kits within the scope of the present invention. Separate formulations of the two components 1 and 2 are described in detail in paragraphs D.2 and D.3 of the present invention. Possible preparations which contain both components 1 and 2 in a single formulation are described for example in paragraph D.4.

The combination of 1 and 2 according to the invention may be administered, within the scope of the abovementioned use and within the scope of the abovementioned process by simultaneously administering the combination of 1 and 2 or, when 1 and 2 are present in different preparations, by administering components 1 and 2 simultaneously or sequentially. The term sequentially within the scope of the present invention refers to any method of administering components 1 or 2 which does not take place simultaneously. By simultaneous administration is meant the method of administration in which at least one of components 1 and 2 is administered for example by infusion over a longer period and the other component is also used during this period. If the two components 1 and 2 are both administered by infusion over a longer period of time, the word simultaneously for the purposes of the present invention means that the infusion periods overlap for at least a short time.

Particularly when treating ischaemic stroke, which is the preferred indication within the scope of the present invention, most preferably when treating acute ischaemic stroke, components 1 and 2 are preferably given simultaneously or at least within a short time of each other, i.e. for example within one hour. Treatment with the drug combinations according to the invention is particularly effective when it is given as quickly as possible after the stroke takes place. Preferably, the treatment starts at the latest within about 6 hours, most preferably within 4 hours, more preferably still within 3 hours after the stroke occurs.

D.2) Pharmaceutical Formulation and Administration of Component 1:

Within the scope of the present invention the compounds 1 may be administered orally, transdermally, by inhalation or parenterally. The compounds 1 occur as active ingredients in conventional preparations, for example in compositions which consist essentially of an inert pharmaceutical carrier and an effective dose of the active substance, such as for example tablets, coated tablets, capsules, lozenges, powders, solutions, suspensions, emulsions, syrups, suppositories, transdermal systems etc.. An effective dose of the compounds 1 , particularly the compounds of formulae 1a and 1b, is between 1 and 1000, preferably between 1 and 500, most preferably between 5-300 mg/dose for oral administration, and between 0.001 and 100, preferably between 0.1 and 70 mg/dose for intravenous, subcutaneous or intramuscular administration.

It is particularly possible to use the component 1 according to the invention as a solution for infusion, preferably in a physiological saline or nutrient saline solution. In an infusion, for example 10-100 mg/h, preferably 20-60 mg/h of the compound 1 may be used.

The compound 1 crobenetine, which is particularly preferred according to the invention, is most preferably administered intravenously by infusion. Preferably between 10 and 60 mg (based on the free base crobenetine) are administered per dose. It is also possible to administer crobenetine as component 1 of the drug combination according to the invention at different time intervals in different doses for each therapy. For example, crobenetine may be given as component 1 first of all in a dosage of 20-70 mg, preferably 30 to 60 mg, most preferably 50 mg over a period of about 30 minutes to 2 hours, preferably over 45 to 90 minutes, most preferably over one hour. This first administration of component 1 crobenetine may then be followed by further administrations in doses of for example 5 to 50 mg, preferably 10 to 40 mg, most preferably 20 to 30 mg over a period of 2 to 8 hours, preferably 3 to 7 hours, most preferably 4 to 6 hours. If desired this second administration of component 1 crobenetine may be followed others.

Some particular embodiments of formulations for parenteral administration are described hereinafter which may be used in particular for the compounds of formulae 1a and 1b which are particularly preferably used as sodium channel blockers 1.

These formulations contain at least one compound of formula 1a or 1b or one of the pharmaceutically acceptable salts thereof and a cyclodextrin derivative, particularly gamma-cyclodextrin (γ-CD), hydroxypropyl-gamma-cyclodextrin (HP-γ-CD), hydroxypropyl-beta-cyclodextrin (HP-β-CD) or sulphobutylether-beta-cyclodextrin (SBE-β-CD). The preferred cyclodextrin derivative is hydroxypropyl-γ-cyclodextrin. Hydroxypropyl-γ-cyclodextrin with a molar substitution level of 0.5 to 0.7 is sold for example by Messrs Wacker-Chemie GmbH, D-Burghausen, under the name “CAVASOL ® W8 HP Pharma”. “CAVASOL ® W8 HP Pharma” is particularly preferred for these pharmaceutical compositions.

Apart from the compound of formula 1a or 1b and the cyclodextrin derivative the pharmaceutical compositions intended for parenteral use according to the invention may contain hydroxy acids such as for example malic acid, lactic acid, tartaric acid or citric acid. They may also optionally contain conventional excipients and carriers such as for example the isotonic agents glucose, mannitol or sodium chloride or sodium acetate or sodium acetate trihydrate as buffer combined with acetic acid or a citric acid/phosphate buffer consisting of e.g. citric acid and disodium hydrogen phosphate or disodium hydrogen phosphate dihydrate. The solvent used is normally water for injections.

The molar ratio of the compound of formula 1a or 1b to cyclodextrin is between 1:1 and 1:5 for example in these formulations. A molar ratio of 1:2.5 to 1:3.5 is preferred. In the presence of hydroxy acid this molar ratio is preferably between 1:0.5 and 1:3 according to the invention; a molar ratio of 1:0.5 to 1:1.5 is particularly preferred. These formulations are most preferably used with crobenetine as component 1.

Apart from the formulations described above containing at least one compound of formula 1a or 1b or one of the pharmaceutically acceptable salts thereof and a cyclodextrin derivative, equally preferred formulations for parenteral use according to the invention are those which contain mannitol as excipient, in addition to a compound of formula 1a or 1b or one of the pharmaceutically acceptable salts thereof. The amount of mannitol is preferably chosen so as to obtain an isotonic solution. These pharmaceutical compositions may optionally also contain other conventional excipients and carriers such as for example an acetic acid/acetate buffer consisting of acetic acid and sodium acetate or sodium acetate-trihydrate or a citric acid/phosphate buffer consisting for example of citric acid and disodium hydrogen phosphate or disodium hydrogen phosphate dihydrate. Usually, the quantity of the buffer components is selected so as to obtain a particular pH value and a particular buffer capacity. The solvent used is normally water for injections.

Preferably, the pharmaceutical composition contains an acetic acid/acetate buffer in addition to the isotonic agent mannitol. A 0.005 to 0.05 molar, preferably a 0.005 to 0.02 molar acetic acid/acetate buffer with a pH of 3.8 to 5 is particularly preferred while a 0.01 molar acetic acid/acetate buffer with a pH value of about 4 is most particularly preferred. The concentration specified relates to the total concentration of acetic acid and acetate; the ratio of acetic acid to acetate results from the desired pH. The pH specified is measured both in the pure buffer solution and in the finished solution for injection of infusion. These formulations are particularly preferably used when crobenetine is used as component 1.

D.2.1) Examples of pharmaceutical
formulations of component 1:

| Formulation Example 1: | | |
|---|---|---|
| crobenetine hydrochloride | 767 | mg |
| HpγCD*) | 10000 | mg |
| mannitol | 11000 | mg |
| acetic acid 99% | 125.25 | mg |
| sodium acetate trihydrate | 56.5 | mg |
| water for injections ad | 250 | ml |
| Formulation Example 2: | | |
| crobenetine hydrochloride | 383.5 | mg |
| γCD | 5000 | mg |
| NaCl | 2250 | mg |
| water for injections ad | 250 | ml |
| Formulation Example 3: | | |
| crobenetine hydrochloride | 767 | mg |
| HPβCD | 7500 | mg |
| mannitol | 12500 | mg |
| acetic acid 99% | 125.25 | mg |
| sodium acetate trihydrate | 56.5 | mg |
| water for injections ad | 250 | ml |
| Formulation Example 4: | | |
| crobenetine hydrochloride | 767 | mg |
| SBEβCD | 5000 | mg |
| mannitol | 12500 | mg |
| acetic acid 99% | 125.25 | mg |
| sodium acetate trihydrate | 56.5 | mg |
| water for injections ad | 250 | ml |
| Formulation Example 5: | | |
| crobenetine hydrochloride | 767 | mg |
| HPγCD | 2500 | mg |
| citric acid | 708 | mg |
| mannitol | 12500 | mg |
| acetic acid 99% | 125.25 | mg |
| sodium acetate trihydrate | 56.5 | mg |
| water for injections ad | 250 | ml |
| Formulation Example 6: | | |
| crobenetine hydrochloride | 767 | mg |
| γCD | 2500 | mg |
| tartaric acid | 138.25 | mg |
| NaCl | 2250 | mg |
| water for injections ad | 250 | ml |
| Formulation Example 7 | | |
| (solution for infusion (acetate buffer pH 4)) | | |
| crobenetine hydrochloride | 274 | mg |
| mannitol | 25000 | mg |
| acetic acid 99% | 250.5 | mg |
| sodium acetate trihydrate | 113.0 | mg |
| water for injections ad | 500 | ml |
| Formulation Example 8 | | |
| (solution for infusion (acetate buffer pH 4.5)) | | |
| crobenetine hydrochloride | 274 | mg |
| mannitol | 25000 | mg |
| acetic acid 99% | 180.0 | mg |
| sodium acetate trihydrate | 265.0 | mg |
| water for injections ad | 500 | ml |
| Formulation Example 9 | | |
| (solution for infusion (acetate buffer pH 4)) | | |
| crobenetine hydrochloride | 383.6 | mg |
| mannitol | 25000 | mg |
| acetic acid 99% | 501.0 | mg |
| sodium acetate trihydrate | 226.0 | mg |
| water for injections ad | 500 | ml |
| Formulation Example 10 | | |
| (solution for infusion (acetate buffer pH 4)) | | |
| crobenetine hydrochloride | 767 | mg |
| mannitol | 11000 | mg |
| acetic acid 9% | 125.25 | mg |
| sodium acetate trihydrate | 56.5 | mg |
| water for injections ad | 250 | ml |
| Formulation Example 11 | | |
| (solution for infusion (acetate buffer pH 4.5)) | | |
| crobenetine hydrochloride | 767 | mg |
| mannitol | 25000 | mg |
| acetic acid 99% | 90.0 | mg |
| sodium acetate trihydrate | 132.5 | mg |
| water for injections ad | 500 | ml |
| Formulation Example 12 | | |
| (solution for infusion (acetate buffer pH 4)) | | |
| crobenetine hydrochloride | 219.1 | mg |
| mannitol | 5000 | mg |
| acetic acid 99% | 50.1 | mg |
| sodium acetate trihydrate | 22.6 | mg |
| water for injections ad | 100 | ml |

*)for example “CAVASOL ® W8 HP Pharma” made by Wacker

The amount of active substance given can be controlled by administering a particular volume of one of the solutions described above. For example the daily administration of 100 ml of a solution according to Example 1 corresponds to a dose of 280 mg of crobenetine a day.

D.3) Pharmaceutical Formulation and Administration of Component 2:

The magnesium salt 2 used within the scope of the combination according to the invention may be given orally or parenterally within the scope of the present invention, parenteral administration being particularly preferred. It may be administered parenterally particularly by intravenous, intraarterial, intramuscular, intra- or subcutaneous injection. Typical formulations are aqueous solutions for infusion or injection, which may optionally contain conventional stabilisers, solubilisers and preservatives as further ingredients.

Typically, within the scope of the present invention, a total of between 30 and 120 mmol, preferably about 50 to 100 mmol, more preferably about 70 to 90 mmol of magnesium are administered per dose. The substance is administered, for example in the form of an infusion which is administered over a period of about 6 to 48 hours, preferably about 12 to 36 hours, more preferably about 18 to 30 hours. Within this period the dose in question can be varied for each time interval. For example in a first interval between about 5 and 25 mmol, preferably between about 10 and 20 mmol of magnesium may be administered over a period of about 5 minutes to 1 hour, preferably over a period of about 10 to 30 minutes and then in a second interval between 25 and 100 mmol, preferably between about 40 and 80 mmol, more preferably between about 50 and 70 mmol of magnesium are administered, for example, over a period of about 5 to 48 hours, preferably about 12 to 36 hours, more preferably about 20 to 28 hours. However, the dosage and administration period may differ from those given above as a guide, depending on the patient and the clinical picture. As a rule it may be desirable to adjust the dosage and period of administration of magnesium so that the plasma levels of magnesium thus produced are above the natural plasma levels of magnesium by about a factor of 1.5 to 2.5, preferably by about a factor of 2. For example, component 2 may also be administered by one or more injections.

D.3.1) Pharmaceutical formulation examples of component 2:

| | | | |
|---|---|---|---|
| a) | Injectable solution: | | |
| | magnesium sulphate | 1000 | mg |
| | water for injections 10 ml | | |
| b) | Injectable solution: | | |
| | magnesium sulphate | 2000 | mg |
| | water for injections 10 ml | | |
| c) | concentrated solution for infusion | | |
| | (for preparing a solution for infusion): | | |
| | magnesium sulphate | 5000 | mg |
| | water for injections 10 ml | | |

In the formulation examples shown above the information relates to magnesium sulphate in the anhydrous form.

D.4) Examples of pharmaceutical
formulations containing components 1 and 2:

| Formulation Example 1 | | |
|---|---|---|
| (solution for infusion (acetate buffer pH 4)) | | |
| crobenetine hydrochloride | 274 | mg |
| mannitol | 25000 | mg |
| acetic acid 99% | 250.5 | mg |
| sodium acetate trihydrate | 113.0 | mg |
| magnesium sulphate | 500 | mg |
| water for injections ad | 500 | ml |
| Formulation Example 2 | | |
| (solution for infusion (acetate buffer pH 4.5)) | | |
| crobenetine hydrochloride | 274 | mg |
| mannitol | 25000 | mg |
| acetic acid 99% | 180.0 | mg |
| sodium acetate trihydrate | 265.0 | mg |
| magnesium sulphate | 500 | mg |
| water for injections ad | 500 | ml |
| Formulation Example 3 | | |
| (solution for infusion (acetate buffer pH 4)) | | |
| crobenetine hydrochloride | 274 | mg |
| mannitol | 25000 | mg |
| acetic acid 99% | 250.5 | mg |
| sodium acetate trihydrate | 113.0 | mg |
| magnesium sulphate | 1000 | mg |
| water for injections ad | 500 | ml |
| Formulation Example 4 | | |
| (solution for infusion (acetate buffer pH 4.5)) | | |
| crobenetine hydrochloride | 274 | mg |
| mannitol | 25000 | mg |
| acetic acid 99% | 180.0 | mg |
| sodium acetate trihydrate | 265.0 | mg |
| magnesium sulphate | 1000 | mg |
| water for injections ad | 500 | ml |
| Formulation Example 5 | | |
| (solution for infusion (acetate buffer pH 4)) | | |
| crobenetine hydrochloride | 274 | mg |
| mannitol | 25000 | mg |
| acetic acid 99% | 250.5 | mg |
| sodium acetate trihydrate | 113.0 | mg |
| magnesium sulphate | 3000 | mg |
| water for injections ad | 500 | ml |
| Formulation Example 6 | | |
| (solution for infusion (acetate buffer pH 4.5)) | | |
| crobenetine hydrochloride | 274 | mg |
| mannitol | 25000 | mg |
| acetic acid 99% | 180.0 | mg |
| sodium acetate trihydrate | 265.0 | mg |
| magnesium sulphate | 3000 | mg |
| water for injections ad | 500 | ml |
| Formulation Example 7 | | |
| (solution for infusion (acetate buffer pH 4)) | | |
| crobenetine hydrochloride | 274 | mg |
| mannitol | 25000 | mg |
| acetic acid 99% | 250.5 | mg |
| sodium acetate trihydrate | 113.0 | mg |
| magnesium sulphate | 5000 | mg |
| water for injections ad | 500 | ml |
| Formulation Example 8 | | |
| (solution for infusion | | |
| (acetate buffer pH 4.5)) | | |
| crobenetine hydrochloride | 274 | mg |
| mannitol | 25000 | mg |
| acetic acid 99% | 180.0 | mg |
| sodium acetate trihydrate | 265.0 | mg |
| magnesium sulphate | 5000 | mg |
| water for injections ad | 500 | ml |

In the formulation examples provided above the information relates to magnesium sulphate in its anhydrous form.

We claim:

1) Drug combinations comprised of one or more sodium channel blocker 1 and one or more magnesium salt 2, optionally in the presence of conventional excipients or carriers.

2) Drug combinations according to claim 1, characterised in that 1 is selected from the group consisting of pirmencol, sipatrigine, irampanel, pilsicainide, oxcarbazepine, topiramate, fosphenytoin, flunarizin, ropivacaine, levobupivacaine, zonisamide, mexiletine, bipridil, bisaramil, milacainide, safinamide, bupivacaine, tetrodotoxin, NS 7, the compounds of general formula 1a 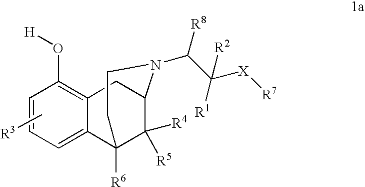
wherein
X denotes a single bond, —O, C₁-C₄-alkylene, an alkylene bridge with 1 to 8 carbon atoms which may be branched or unbranched and may have at any point in the bridge one or two oxygen atom(s) or a nitrogen atom, preferably O—C₁-C₃-alkylene or —O—CH₂—CH₂—O—, —O—CH₂—CH₂—NH—;
R¹ denotes hydrogen, methyl, ethyl, phenyl;
R² denotes hydrogen, methyl;
R³ denotes hydrogen, fluorine, chlorine, bromine, hydroxy, methyl, methoxy;
R⁴ denotes hydrogen, methyl, ethyl;
R⁵ denotes hydrogen, methyl, ethyl;
R⁶ denotes hydrogen, methyl, ethyl;
R⁷ denotes tert.-butyl, cyclohexyl or phenyl, while phenyl may optionally be substituted by R⁹ and R¹⁰, which may be identical or different;
R⁸ denotes hydrogen, C₁-C₄-alkyl;
R⁹ denotes hydrogen, methyl, fluorine, chlorine, bromine, methoxy;
R¹⁰ denotes hydrogen, methyl, fluorine, chlorine, bromine, methoxy;
optionally in the form of the individual optical isomers, mixtures of the individual enantiomers or racemates as well as in the form of the free bases or the corresponding acid addition salts with pharmacologically acceptable acids;
and the compounds of general formula 1b 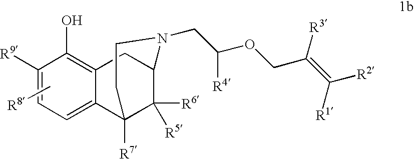
wherein
R^{1′}, R^{2′} and R^{3′} which may be identical or different, denote hydrogen, methyl or ethyl;
R^{4′} denotes hydrogen, methyl or ethyl;
R^{5′}, R^{6′} and R^{7′} which may be identical or different, denote hydrogen, methyl or ethyl;
R^{8′} and R^{9′} which may be identical or different, denote hydrogen, fluorine, chlorine, bromine, methyl, ethyl, hydroxy or methoxy,
optionally in the form of the racemates, the enantiomers, the diastereomers and the mixtures thereof, and optionally the pharmacologically acceptable acid addition salts thereof.

3) Drug combinations according to claim 2, characterised in that 1 is selected from the group consisting of pirmencol, pilsicainide, sipatrigine, irampanel, fosphenytoin, zonisamide, mexiletine, bipridil, bisaramil, milacainide, NS 7, the compounds of general formula 1a wherein
X denotes C₁-C₃-alkylene, —O—CH₂—CH₂—O— or —O—CH₂—CH₂—NH—;
R¹ denotes hydrogen or methyl;
R² denotes hydrogen or methyl;
R³ denotes hydrogen or chlorine;
R⁴ denotes hydrogen or methyl;
R⁵ denotes hydrogen or methyl;
R⁶ denotes methyl or ethyl;
R⁷ denotes tert.-butyl, cyclohexyl or phenyl, while phenyl may optionally be substituted by R⁹ and R¹⁰, which may be identical or different;
R⁸ denotes hydrogen;
R⁹ denotes hydrogen, methyl, fluorine or chlorine;
R¹⁰ denotes hydrogen, methyl, fluorine or chlorine;
optionally in the form of the individual optical isomers, mixtures of the individual enantiomers or racemates as well as in the form of the free bases or the corresponding acid addition salts with pharmacologically acceptable acids;
and the compounds of general formula 1b, wherein
R^{1′}, R^{2′} and R^{3′} which may be identical or different, denote hydrogen or methyl;
R^{4′} denotes hydrogen or methyl;
R^{5′}, R^{6′} and R^{7′} which may be identical or different, denote hydrogen or methyl, preferably methyl;
R^{8′} denotes hydrogen, methyl, hydroxy or methoxy, preferably hydrogen or methyl,
R^{9′} denotes hydrogen or methyl,
optionally in the form of the racemates, the enantiomers, the diastereomers and the mixtures thereof, and optionally the pharmacologically acceptable acid addition salts thereof.

4) Drug combinations according to claim 1, characterised in that 2 is selected from the list consisting of [magnesium adipate, magnesium-L-aspartate, magnesium carbonate, magnesium-L-hydrogenaspartate, magnesium hydrogencitrate, magnesium hydrogenglutamate, magnesium sulfate, magnesium chloride, trimagnesium dicitrate and magnesium acetate].

5) Drug combinations according to claim 4, characterised in that 2 is selected from the list consisting of magnesium sulfate, magnesium chloride and magnesium acetate.

6) Drug combinations according to claim 1, characterised in that the active ingredients 1 and 2 are contained in a single, or in two separate, preferably in two separate preparations.

7) A method of treating ischaemic conditions which comprises administering to a patient in need thereof a therapeutically effective amount of a drug combination according to claim 1.

8) A method according to claim 7, for the treatment of cardiac or cerebral ischaemias or the treatment of stroke.

9) Use of one or more sodium channel blocker 1 for preparing a pharmaceutical composition for the combined treatment of ischaemic conditions of various origins with one or more magnesium salt 2.

10) Use according to claim 9, characterised in that 1 is selected from among the compounds according to claim 2 and further characterised in that 2 is selected from among the compounds according to claim 4.

11) Use of one or more sodium channel blocker 1 according to claim 9, wherein said magnesium salt is selected from the list consisting of [magnesium adipate, magnesium-L-aspartate, magnesium carbonate, magnesium-L-hydrogenaspartate, magnesium hydrogencitrate, magnesium hydrogenglutamate, magnesium sulfate, magnesium chloride, trimagnesium dicitrate and magnesium acetate].
